(19) United States
(12) Patent Application Publication
(10) Pub. No.: US 2006/0263398 A1
(43) Pub. Date: Nov. 23, 2006

(54) Method and compound for skin chemical peel

(75) Inventor: Abdala Kalil, Miami Beach, FL (US)

Correspondence Address: Alan M. Weisberg;Christopher & Weisberg, P.A., Suite 2040, 200 East Las Olas Boulevard, Fort Lauderdale, FL, 33301, US

(73) Assignee: Kalil Enterprises, L.L.P.

(21) Appl. No.: 11/134,887

(22) Filed: May 23, 2005

(51) Int. Cl. A61K 8/36

(52) U.S. Cl. 424/401

(57) ABSTRACT

The present invention provides a skin peel composition including a plurality of keratolytic substances, such as trichloroacetic acid and salicylic, and a post inflammatory hyper pigmentation reducing substance such as retinoic acid. In addition, the skin peel composition may further include ascorbic acid (vitamin C), as well as vitamins A and E. Application of the skin peel composition may be either topically in the form of a cream or gel, and may further be injected subcutaneously into the skin to achieve deeper penetration of the composition within the skin layers of the area to be treated.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a dermatological composition and method for application thereof, and in particular a composition for efficiently and effectively performing chemical peel procedures.

### BACKGROUND OF THE INVENTION

A variety of skin problems including acne, scars, pigmentation disorders, sun exposure and the like have led to the development of numerous compositions and therapies directed toward their treatment. Such treatments may include skin creams, lotions, or ointments which are applied to help rejuvenate or nourish the underlying skin layers. An additional treatment includes the application of a “skin peel” compound, which aids in the removal of damaged skin layers.

Skin peels generally include one or more acidic components, such as trichloroacetic acid (TCA) or salicylic acid, which are applied to the skin in order to remove a layer of the skin. The removal of a layer of skin allows a new layer of skin lining to develop, often resulting in evenly colored, healthier skin and further reducing the skin problems identified above. Depending on the level of existing skin problems a patient may have, increased acidic concentrations or prolonged application of a skin peel composition may be implemented in order to treat deeper skin layers or severe cases of skin disorders.

Unfortunately, skin peels may have adverse side effects. Depending on the particular acidic component used, the removal of a skin layer may result in post inflammatory hyper pigmentation (PIH), which appears as variations in skin pigmentation, i.e., lighter or darker patches on the skin area treated. In order to prevent or reduce such pigmentation complications, the skin area to be treated may require several applications of additional preparatory compositions prior to the application of the skin peel itself. Such repeated preparatory applications may extend over many weeks before the skin can finally be subjected to the skin peel in an attempt to minimize the likelihood of experiencing pigmentation abnormalities. Furthermore, subsequent to being treated, a patient may wish to reduce social interaction due to the appearance of the peeling skin in the days following the skin peel application. Additionally, depending on the depth of treatment, the patient may even experience some pain and discomfort. The combination of the preparation of the skin prior to the application of the skin peel and the down-time resulting after treatment prolongs the amount of time required for a successful skin peel treatment, thereby burdening and inconveniencing a patient.

In view of the above disadvantages, it would be desirable to provide a skin peel composition applicable using a number of delivery and treatment techniques, which reduces pain and discomfort, substantially reduces any preparation time or down-time, and further provides therapeutic results faster than that of known skin peel compositions.

### SUMMARY OF THE INVENTION

In accordance with one aspect, the present invention provides a skin peel composition having a plurality of keratolytic substances as well as a PIH reducing substance. The plurality of keratolytic substances may include trichloroacetic acid and salicylic acid, while the PIH reducing substance can include retinoic acid. In addition, the skin peel composition may further include ascorbic acid (vitamin C), as well as vitamins A and E.

In accordance with another aspect, the present invention provides a method for treating skin using a skin peel composition in which a skin peel composition is created. The skin peel composition has trichloroacetic acid, salicylic acid and retinoic acid. Once the skin peel composition has been created, the composition is then applied to a predetermined area of skin. In addition, application of the skin peel composition may be either topically in the form of a cream or gel, and may alternatively provide for subcutaneous injection directly into the skin to achieve deeper penetration of the composition within the skin layers of the area to be treated.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a flow chart of a skin care treatment method performed in accordance with the present invention; and

FIG. 2 is a flow chart of an alternative method of skin care treatment performed in accordance with the present invention.

### DETAILED DESCRIPTION OF THE INVENTION

The present invention provides a skin peel composition having three primary components, including trichloroacetic acid (TCA), salicylic acid, and retinoic acid. In addition to these components, additional ingredients may include vitamins, minerals, and other agents providing therapeutic benefits to the treated skin area.

TCA is an acidic substance which is used in skin peeling processes. While the concentration needed for a particular application of the composition of the present invention may vary depending on the depth of treatment for a particular patient, the composition of the present invention preferably contains a TCA concentration between 1% and 14% by weight per unit volume.

The skin peel composition further includes salicylic acid, which provides keratolytic effects to applied areas of skin, resulting in the destruction, and thus peeling, of the skin layer exposed to the acid. The skin peel composition according to the present invention preferably includes a salicylic acid concentration between 1% and 14% by weight per unit volume.

Retinoic acid (commonly known as Retin-A®) can be employed to combat skin disorders such as acne and has also been used to reduce the appearance of wrinkles and aging in the skin due to prolonged sun exposure. However, retinoic acid also appears to inhibit melatonin synthesis, which is an underlying cause of post inflammatory hyper pigmentation (PIH). PIH often appears subsequent to skin peel procedures as blotchy skin, i.e., areas of the skin surface having variations in pigment. As such, retinoic acid is preferably present in the composition of the present invention in a concentration between 0.01% and 0.4% by weight.

The skin peel composition of the present invention also preferably includes ascorbic acid (vitamin C) in a concentration between 0.5% and 3.25% by weight. Ascorbic acid, once absorbed by the skin, has been shown to stimulate collagen synthesis and provide protection against sunlight. Moreover, ascorbic acid is an antioxidant which further helps to protect skin by neutralizing free radicals, which would otherwise destroy the skin and its components. Vitamin E can be included in the skin peel composition as a skin-protective ingredient that further promotes skin healing. The concentration of vitamin E is preferably between 0.01% and 0.2% by weight. In addition, vitamin A may be included to further promote skin healing and protection in a concentration preferably between 1000 IU/ml and 3000 IU/ml.

The toxic acidic nature of TCA, when used solely in higher concentrations, can cause significant damage and inflammation to the skin area treated. Moreover, even when used in lower concentrations, TCA has commonly caused uneven peeling and blotchy skin appearances when used as a peeling agent. Salicylic acid also can prove caustic to skin areas when used in higher concentrations. However, when the two acids are combined, along with the retinoic acid, the skin peel composition of the present invention advantageously provides a synergistic effect in that the acids provide a deeper penetration of the skin peel, thereby allowing the retinoic acid to interact with deeper layers of skin tissue to prevent the pigmentation abnormalities potentially caused by TCA or salicylic acid alone. Furthermore, by combining the PIH-inhibiting retinoic acid with TCA and salicylic acid in a single skin peel composition to be applied, the preparation time typically required in order to prevent PIH is substantially reduced and even potentially eliminated. Because of the synergistic effects of the combined ingredients, the additional therapeutic ingredients, such as vitamins A, C, and E, can penetrate deeper into the tissue as well, resulting in an enhanced level of absorption into the skin.

While the skin peel composition has been described as containing the components listed above, the composition may, of course, further comprise additional components such as preservatives, stabilizers, additional antioxidants, surfactants, pigments, fragrances or other adjuvants.

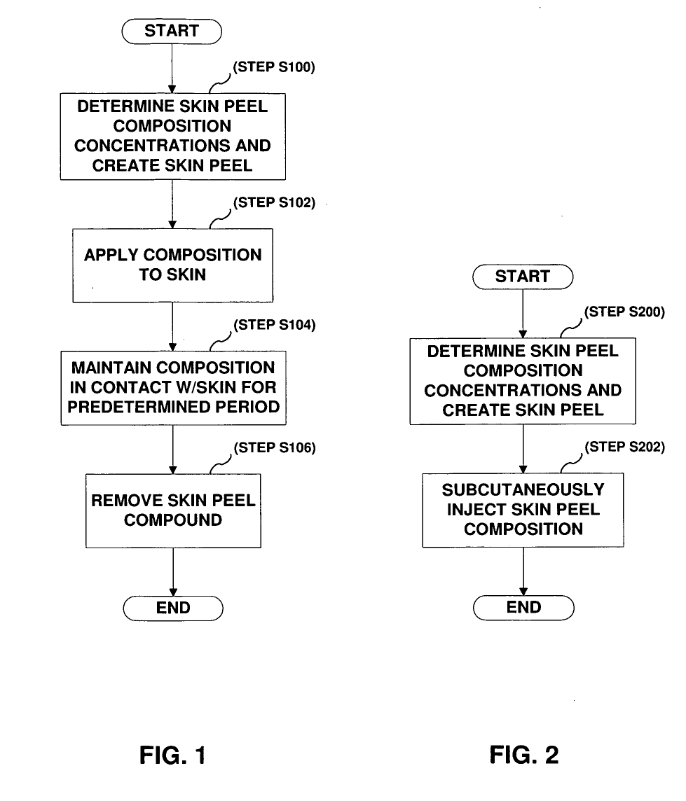

In an exemplary method of use, the skin peel composition of the present invention can be in the form of a cream or gel-like solution which is topically applied to the skin area to be treated. As illustrated in FIG. 1, a determination of concentrations of the individual components of the skin peel composition is made, which may be varied within the ranges specified above depending on the depth of skin penetration desired or the amount of damage that is to be repaired (Step S100). Upon a determination of the appropriate quantities of each component to be used in the composition, the skin peel composition is applied to a predetermined skin surface area (Step S102). The skin peel composition then remains in contact with the predetermined skin surface for a predetermined period of time (Step S104). Similarly to modifying the concentrations of the ingredients, the length and/or frequency of applications of the skin peel may also be altered according to the desired level of therapy to be provided. Once the composition has been in contact with the skin for the desired period of time, the skin peel is removed from the predetermined skin area (Step S106).

As shown in FIG. 2, in addition to the skin peel composition being applied topically, the present invention also provides for the skin peel composition to be applied directly to underlying skin layers by the use of a subcutaneous injection, a method similar to that used in mesotherapy. In this alternative method, a determination of concentrations of the individual components of the skin peel composition is made (Step S200). Upon a determination, the skin peel composition is injected into the skin area to be treated (Step S202). Subcutaneous injection of the skin peel composition provides direct contact with the underlying dermal layers, thereby providing an increased depth of affected tissue and an even faster rate of treatment than that achieved through the external skin application method of treatment. Moreover, when the composition is injected, determining the length of time the composition is to be applied and the subsequent removal of the composition from the skin are no longer required, thereby easing the application and use of the composition.

The present invention advantageously provides a composition for a chemical skin peel and a method for application thereof, which reduces pain and discomfort, substantially reduces preparation and down-time, and further provides therapeutic results faster than that of known skin peel compositions.

It will be appreciated by persons skilled in the art that the present invention is not limited to what has been particularly shown and described herein above. A variety of modifications and variations are possible in light of the above teachings without departing from the scope and spirit of the invention, which is limited only by the following claims.

What is claimed is:

1. A skin peel composition, comprising:
a plurality of keratolytic substances; and
a PIH reducing substance.

2. The skin peel composition according to claim 1, wherein the plurality of keratolytic substances includes between substantially 1% and substantially 14% by weight per unit volume of trichloroacetic acid.

3. The skin peel composition according to claim 2, wherein the plurality of keratolytic substances includes between substantially 1% and substantially 14% by weight per unit volume of salicylic acid.

4. The skin peel composition according to claim 3, wherein the PIH reducing substance includes between substantially 0.01% and substantially 0.4% by weight of retinoic acid.

5. The skin peel composition according to claim 4, further comprising between substantially 0.5% and substantially 3.25% by weight of ascorbic acid.

6. The skin peel composition according to claim 4, further comprising between substantially 0.01% and substantially 0.2% by weight of vitamin E.

7. The skin peel composition according to claim 4, further comprising between substantially 1000 IU/mL and substantially 3000 IU/mL of vitamin A.

8. A skin peel composition, comprising:
between substantially 1% and substantially 14% by weight per unit volume of trichloroacetic acid;
between substantially 1% and substantially 14% by weight per unit volume of salicylic acid;
between substantially 0.01% and substantially 0.4% by weight of retinoic acid;
between substantially 0.5% and substantially 3.25% by weight of ascorbic acid;
between substantially 0.01% and substantially 0.2% by weight of vitamin E; and
between substantially 1000 IU/mL and substantially 3000 IU/mL of vitamin A.

9. The skin peel composition according to claim 8, wherein the skin peel composition is included in a topical compound.

10. The skin peel composition according to claim 8, wherein the skin peel composition is included in a subcutaneously injectable compound.

11. A method for treating skin, comprising:
creating a skin peel composition comprising trichloroacetic acid, salicylic acid and retinoic acid; and
applying the skin peel composition to a predetermined area of skin.

12. The method for treating skin according to claim 11, further comprising:
maintaining the skin peel composition in contact with the predetermined area of skin for a predetermined period of time; and
removing the skin peel composition from contact with the predetermined area of skin.

13. The method for treating skin according to claim 11, wherein the concentration of trichloroacetic acid is between substantially 1% and substantially 14% by weight per volume of the skin peel composition.

14. The method for treating skin according to claim 11, wherein the concentration of salicylic acid is between substantially 1% and substantially 14% by weight per volume of the skin peel composition.

15. The method for treating skin according to claim 13, wherein the concentration of retinoic acid is between substantially 0.01% and substantially 0.4% by weight of the skin peel composition.

16. The method for treating skin according to claim 11, wherein the skin peel composition further comprises ascorbic acid, vitamin E, and vitamin A.

17. The method for treating skin according to claim 12, wherein application is made to an external surface of skin.

18. The method for treating skin according to claim 11, wherein application is made subcutaneously.

19. The method for treating skin according to claim 15, wherein application is made to an external surface of skin.

20. The method for treating skin according to claim 15, wherein application is made subcutaneously.
